SEQUENCE LISTING (30 sequences)

SEQ ID NO: 1 - protein, 114 aa, Homo sapien
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 231 aa, Homo sapien
PKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 346 aa, artificial sequence
  fusion protein
NWVNVISDLKKTEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 347 aa, artificial sequence
  fusion protein
NWVNVISDLKKTEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - DNA, 341 bp, Homo sapien
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttc

SEQ ID NO: 7 - DNA, 697 bp, Homo sapien
cccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgat

SEQ ID NO: 8 - DNA, 700 bp, Mus musculus
cccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgag

SEQ ID NO: 9 - DNA, 1047 bp, artificial sequence
  DNA coding for fusion protein
aactgggtgaatgtaataagtgatttgaaaaaaaccgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 10 - DNA, 1045 bp, artificial sequence
  DNA coding for fusion protein
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcggatcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgag

SEQ ID NO: 11 - DNA, 63 bp, Homo sapien
atggagacagacacactcctgctatgggtactgctgctctgggttccaggttccactggtgac

SEQ ID NO: 12 - DNA, 72 bp, Homo sapien
atgcccatggggtctctgcaaccgctggccaccttgtacctgctggggatgctggtcgcttcctgcctcgga

SEQ ID NO: 13 - DNA, 75 bp, Homo sapien
atgaaccggggagtcccttttaggcacttgcttctggtgctgcaactggcgctcctcccagcagccactcaggga

SEQ ID NO: 14 - DNA, 60 bp, Homo sapien
atgtacaggatgcaactcctgtcttgcattgcactaagtcttgcacttgtcacaaacagt

SEQ ID NO: 15 - DNA, 69 bp, Homo sapien
atgaaagtctctgccgcccttctgtgcctgctgctcatagcagccaccttcattccccaagggctcgct

SEQ ID NO: 16 - DNA, 40 bp, Homo sapien
atgtcttcattttgggctgtttcagtgcagggcttcctaa

SEQ ID NO: 17 - DNA, 144 bp, Homo sapien
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagcc

SEQ ID NO: 18 - DNA, 74 bp, artificial sequence
  oligonucleotide
ctagccaccatggagacagacacactcctgctatgggtactgctgctctgggttccaggttccactggtgacaa

SEQ ID NO: 19 - DNA, 74 bp, artificial sequence
  oligonucleotide
ccagttgtcaccagtggaacctggaacccagagcagcagtacccatagcaggagtgtgtctgtctccatggtgg

SEQ ID NO: 20 - DNA, 36 bp, artificial sequence
  oligonucleotide
ctgggtgaatgtaataagtgatttgaaaaaaattga

SEQ ID NO: 21 - DNA, 37 bp, artificial sequence
  oligonucleotide
gatcttcaatttttttcaaatcacttattacattcac

SEQ ID NO: 22 - DNA, 111 bp, artificial sequence
  oligonucleotide
ctagccaccatggagacagacacactcctgctatgggtactgctgctctgggttccaggttccactggtgacaactgggtgaatgtaataagtgatttgaaaaaaattgaa

SEQ ID NO: 23 - DNA, 111 bp, artificial sequence
  oligonucleotide
ggtggtacctctgtctgtgtgaggagcatacccatgacgacgagacccaaggtccaaggtgaccactgaagacccacttacattattcactaaactttttttaacttctag

SEQ ID NO: 24 - protein, 347 aa, Homo sapien
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 25 - protein, 347 aa, artificial sequence
  mutated Fc
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVQMFINTSDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - DNA, 1108 bp, Homo sapien
atggagacagacacactcctgctatgggtactgctgctctgggttccaggttccactggtgacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgag

SEQ ID NO: 27 - DNA, 1108 bp, artificial sequence
  nucleic acid for mutated Fc
atggagacagacacactcctgctatgggtactgctgctctgggttccaggttccactggtgacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtccaaatgttcatcaacacttcggatcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgag

SEQ ID NO: 28 - DNA, 25 bp, artificial sequence
  Primer
attgaagatcttattcaatctatgc

SEQ ID NO: 29 - DNA, 56 bp, artificial sequence
  primer
ggatccgaagtgttgatgaacatttggacaatatgtacaaaactctgcaaaaattc

SEQ ID NO: 30 - DNA, 29 bp, artificial sequence
  primer
gggatccgaagtgttgatgaacatttgga

(19) United States
(12) Patent Application Publication
(10) Pub. No.: US 2006/0236411 A1
(43) Pub. Date: Oct. 19, 2006

(54) Antagonists il-15

(76) Inventors: Ingeborg Dreher, Mannheim (DE); Thomas Moll, San Diego, CA (US)

Correspondence Address: CLARK & ELBING LLP, 101 FEDERAL STREET, BOSTON, MA, 02110, US

(21) Appl. No.: 10/530,987

(22) PCT Filed: Oct. 13, 2003
(86) PCT No.: PCT/CH03/00666

§ 371 Date: Feb. 15, 2006

(30) Foreign Application Priority Data
Oct. 14, 2002 (EP) 02022869.8

(51) Int. Cl. A01K 67/027; C07H 21/04; C12P 21/04; C07K 14/54; C07K 16/46; C12N 5/06

(52) U.S. Cl. 800/14; 530/351; 530/391.1; 435/69.7; 435/320.1; 435/328; 536/23.53

(57) ABSTRACT

The invention relates to fusion proteins consisting of a wild-type IL-15 and a IgG-Fc fragment, apart from a mouse IgG2b fragment, nucleic acids encoding said proteins, vectors, modified cells, and also to the use thereof for preparing drugs which are used, for example for preventing and/or curing disorders resulting from a transplantation and/or autoimmune diseases.

The invention relates to fusion proteins which are composed of a wild-type IL-15 and an IgG Fc fragment and to their preparation and use for inhibiting immune reactions and for the prophylaxis and/or therapy of transplantation sequelae and/or autoimmune diseases.

An effective immune response is initiated by T cells of the immune system being activated, with this activation being induced by an antigen or mitogen. The activation of the T cells requires a large number of cellular changes, including, for example, the expression of cytokines and their receptors. These cytokines include, inter alia, IL-15 and IL-2.

IL-15 and IL-2 are known growth factors which play a significant role in the proliferation and differentiation of human and murine T cells, macrophages, natural killer (NK) cells, cytotoxic T cells (CTL), and lymphocyte-activated killer (LAK) cells as well as in the costimulation of B cells which have been activated, for example, by antiimmunoglobulin (anti-IgM) or phorbol esters. The proliferation of these cells augments the immune response of an organism.

IL-15 was described for the first time as a secretory cytokine which induces the proliferation of IL-2-dependent murine cytotoxic T cells (CTLL-2). IL-15 was characterized as being a precursor protein of 162 amino acids in length having a 48-amino acid leader sequence, that is a mature protein of 114 amino acids in length (Grabstein et al., (1994) Science 264(5161):965-8).

IL-15 is formed in epithelial and fibroblast cell lines as well as peripheral blood monocytes. Its specific mRNA has also been found in placenta, skeletal muscles and kidneys (Grabstein et al., see above).

In addition to the biological properties which they have in common, IL-15 and IL-2 also possess homologous structures. Both molecules bind to at least three separate receptor subunits on the membrane of T cells, with the beta and gamma subunit complexes by way of which the signal transduction takes place being the same whereas the alpha subunit is specific for binding IL-15 or IL-2. It has been found that antibodies which are directed against the alpha subunit of the IL-2 receptor do not exert any effect on the binding of IL-15 to its specific alpha subunit (Grabstein et al., see above), whereas antibodies which are directed against the beta subunit of the IL-2 receptor block the activity of IL-15 (Giri et al., (1994) EMBO J., 13:2822). The signal transduction takes place by way of the IL-15 beta and gamma subunits.

In a large number of diseases, it is necessary, for therapeutic reasons, to suppress a response of the patient's immune system. These diseases include, for example, autoimmune diseases, in particular diabetes mellitus type I (Botazzo, G. F., et al., (1985) N Engl J Med 113:353), rheumatoid arthritis, multiple sclerosis, chronic liver diseases, inflammatory intestinal diseases, graft-versus-host disease [GVHD] and transplant rejection (Sakai et al., (1998) Gastroenterology, 114(6):1237-1243; Kivisakk et al., (1998) Clin Exp Immunol, 111(l):193197).

If immunocompetent cells are transferred from a genetically nonidentical organism, these cells then react against the recipient organism (GVHD) (Janeway C. A. and Travers P., Spektrum-Verlag, German edition, 1995, p. 467).

The transplantation of organs or tissues has become a standard method in the case of many life-threatening diseases and, in a large number of cases, has become the only life-saving treatment. However, difficulties exist with regard to rejection reactions in the recipient organism, with these reactions being provoked by immune responses to the foreign cell surface antigens of the transplant.

The degree to which a transplant is rejected in connection with a transplantation depends on the magnitude of the histogenetic difference between the donor and the recipient (histocompatibility). Differences in the antigen patterns exhibited by the donor and recipient organisms induce an immune reaction in the latter, resulting in a rejection reaction directed against the transplant. A transplant is rejected as a result of both humoral and cellular reactions. Humoral effectors are antibodies of differing specificity, such as antibody-dependent cell-mediated cytotoxicity and antibodies which are directed against structures in the donor HLA system. Cellular effectors are, in particular, cytotoxic T cells in combination with macrophages, inter alia (Immunologie [Immunology], Janeway C. A. and Travers P., Spektrum-Verlag, German edition 1995, pp. 522-8).

One therapeutic approach is that of using immunosuppressants, in particular antagonistic IL-15 or IL-2 antibodies, or IL-15 or IL-2 antagonists, to suppress the humoral or the cellular immune response.

A variety of therapies using antibodies directed against IL-15 or IL-2 molecules have been described. Thus, it was possible, for example, to extend the survival time of an allotransplanted primate heart by administering the monoclonal antibody anti-IL-2.beta (Mik.beta-1) (Tinubu et al., (1994) J Immunol. 153:4330). Using monoclonal antibodies directed against the T cell-specific antigen CD3 to block transplant rejection has also been described (Mackie et al., (1990) TransPlantation 49:1150).

Furthermore, a large number of IL-15 antagonists which alter the behavior of IL-15 with regard to binding to its receptor have been described. These antagonists were obtained by introducing (a) mutation(s) into the wild-type IL-15 sequence. Thus, a mutation at amino acid position 56 (aspartate) [position 8 after the leader sequence has been eliminated] which resulted in binding to the alpha subunit of the IL-15 receptor but which prevented binding to the beta subunit has, for example, been described (WO 96/26274). In another approach, a mutation at amino acid position 156 (glutamine) [position 108 after the leader sequence has been eliminated] inhibited interaction with the gamma subunit (WO 96/26274; WO 97/41232). Furthermore, while PEGylated IL-15 permitted binding to the alpha subunit, binding to the beta subunit was no longer possible for steric reasons (Pettit et al., (1997) J Biol Chem, 272 4: 2312-2318).

The above-described IL-15 antagonists are mutated IL-15 (mut-IL-15) sequences which achieved antagonistic effects either on their own or as fusion proteins. These fusion proteins are polypeptides which consist of a N-terminal mut-IL-15 fragment and a C-terminal Fc fragment, in particular a murine IgG2a or human IgG1 (WO 97/41232; Kim et al., (1998) J Immunol., 160:5742-5748).

An Fc (Fragment crystallizable) fragment is to be understood as meaning the fragment of an antibody which does not bind any antigens. The other two identical Fab (fragment antigen binding) fragments of an antibody possess antigen-binding activity (Immunologie [Immunology], Janeway C. A. and Travers P., German edition (1995), p. 117 ff).

However, a disadvantage of these mutated IL-15 molecules is that they possess a primary, secondary and tertiary structure which is altered as compared with that of the wild type IL-15 and, as a result, possess different degradation points, resulting in the appearance of degradation products which do not naturally occur in the cells and which may display a toxic effect in the organism. The nature and extent of these and other side effects are not foreseeable in detail.

Another disadvantage is that patients who are carrying transplants as a rule retain these transplants for their lifetime, which means that they need to ingest immunosuppressants for the whole of their lives. Due to the fact, in particular, that our understanding of the side effects of the long-term intake of these immunosuppressants is inadequate, there is a pressing need to exclude these side effects or at least limit them.

It has been demonstrated that, when immunosuppressive components such as A cyclosporins, FK506 and rapamycin are administered, these agents inhibit the proliferation of all T cells (Penn, (1991) Transplant Proc, 23:1101; Beveridge et al., (1984) Lancet 1:788).

A serious disadvantage is that the administration, which is as a rule systemic, of these immunosuppressants leads to the latter being distributed throughout the entire organism and does not ensure local presence at the site of the transplanted cell(s), tissue or organ. However, inhibiting T cell proliferation throughout the entire organism can give rise to infections, toxic breakdown products or even cancer.

The object of the present invention is, therefore, to produce an immunosuppressant which does not display any, or scarcely any, side effects in an organism in which an immune response is to be inhibited.

It is known that mutated IL-15 molecules, or fusion proteins which comprise a mut-IL-15 and an Fc fragment, exhibit an antagonistic effect on IL-15 by inhibiting or altering receptor binding behavior.

However, it was completely surprising that a fusion protein comprising an N-terminal wild-type IL-15 and a C-terminal Fc fragment, in particular a murine IgG2a, also displays an antagonistic effect even though an agonistic effect would, per se, have been expected. It was only by attaching an Fc fragment to a naturally occurring IL-15 molecule, which is normally immunostimulatory, that it was possible to reverse the mechanism of action, that is achieve inhibition of an immune response.

This finding was surprising precisely because it was not possible, on the assumption that the wild-type IL-15 segment of the fusion protein was folded naturally, to assume that the attached Fc fragment could, on its own, alter the receptor binding behavior such that the entire wild-type IL-15-Fc molecule would display an antagonistic effect with regard to the wild-type IL-15.

Part of the subject matter of the invention therefore relates to a fusion protein which is composed of a wild-type IL-15, on the one hand, and, on the other hand, an IgG Fc fragment, apart from a murine IgG2b Fc fragment.

A fusion protein according to the present invention is to be understood as being the expression product of a fused gene. A fused gene arises from the linking of two or more genes or gene fragments, resulting in the formation of a new combination.

A wild-type IL-15 in accordance with the present invention is understood as meaning the naturally occurring IL-15, as described, for example, in Grabstein et al., (1994) Science 264(5161):965-8, or allelic variants thereof.

An Fc (Fragment cristallizable) fragment is to be understood as being the fragment of an antibody which does not bind any antigens, for example an antibody molecule which lacked the variable domains or else partially or completely lacked the first constant domain of the heavy and light chains. The Fc fragment can be derived from a natural source, be prepared recombinantly and/or be synthesized. The skilled person is familiar with appropriate methods.

The Fc fragment of the fusion protein according to the invention is an immunoglobulin G (IgG) and, specifically, a human or murine IgG1, a human IgG2, a murine IgG2a, a human or murine IgG3 or a human IgG4, preferably a human IgG1 or a murine IgG2a, in particular an IgG1. Preference was given to using the IgGs from the hinge region and downwards. The flexible region in the Ig molecule is designated the hinge region.

IgGs according to the invention are to be understood, for example, as being the following described IgGs:
human IgG1 (Paterson, T. et al., (1998), Immunotechnology 4(1):37-47, murine IgG2a (Sikorav, J. L., (1980), Nucleic Acids Res. 8(14):3143-3155), murine IgG1 (French et al., (1991), J. Immunol. 146(6):2010-2016, human IgG2 (Krawinkel, U. and Rabbitts, T. H., (1982), EMBO J. 1(4):403-407; Wang et al., (1980), J. Immunol. 125(3):1048-1054), murine IgG2b (Schlomchik, M. J., (1987), Nature 328, 805-811), human IgG3 (Huck, S. et al., (1986), Nucleic Acids Res. 14(4):1779-1789), murine IgG3 (Wels et al., (1984), EMBO J., 3(9):2041-2046) and human IgG4 (Pink et al., (1970), Biochem. J., 117(1):33-47) have been described.

The fusion protein according to the invention is preferably a chimeric fusion protein, for example containing a wild-type IL-15 and a heterologous IgG1 Fc fragment or a heterologous IgG2a Fc fragment.

In preferred embodiments, the fusion protein according to the invention comprises the amino acid sequences SEQ ID NO:1, SEQ ID NO:2, SEQ ID NO:3, SEQ ID NO:4 or SEQ ID NO:5.

A further part of the subject matter of the invention relates to a nucleic acid which encodes a fusion protein which contains a wild-type IL-15, on the one hand, and, on the other hand, contains an IgG Fc fragment apart from a murine IgG2b Fc fragment.

The nucleic acid according to the invention preferably encodes a wild-type IL-15 and a human or murine IgG1, a human IgG2, a murine IgG2a, a human or murine IgG3 or a human IgG4, particularly preferably a human IgG1 or a murine IgG2a, most preferably an IgG1.

The nucleic acid according to the invention preferably encodes a fusion protein having one of the amino acid sequences SEQ ID NO:1, SEQ ID NO:2, SEQ ID NO:3, SEQ ID NO:4 or SEQ ID NO:5.

In preferred embodiments, the nucleic acid according to the invention contains the DNA sequences SEQ ID NO:6, SEQ ID NO:7, SEQ ID NO:8, SEQ ID NO:9 or SEQ ID NO:10.

Within the meaning of the present invention, a nucleic acid is understood as being an RNA or DNA, in particular genomic DNA, cDNA or synthetic DNA, which has, for example, been synthesized at the level of phosphoramidation. Combinations and/or modifications of nucleotides of these nucleic acids are likewise encompassed. This term furthermore encompasses single-stranded and double-stranded nucleic acids.

It also encompasses nucleic acids which comprise functionally linked components, for example one or more fused genes, or active parts thereof, encoding one or more fusion proteins according to the invention and also regulatable elements and/or regulatory nucleotide sequences which influence the expression of the gene(s) quantitatively and/or in a time-dependent manner.

Examples of regulatable elements are promoters for constitutive or cell-specific or tissue-specific expression.

Regulatory nucleotide sequences comprise, for example, leader sequences, polyadenylation sequences, for example an SV40 polyadenylation signal, enhancer sequences, IRES sequences and introns.

The leader sequences which are listed below are examples of preferred leader sequences of the present invention:

Igk Leader:

5′-ATGGAGACAGACACACTCCTGCTATGGGTACTGCTGCTCTGGGTTCC
AGGTTCCACTGGTGAC-3′,

CD5 Leader:

5′-ATGCCCATGGGGTCTCTGCAACCGCTGGCCACCTTGTACCTGCTGGG
GATGCTGGTCGCTTCCTGCCTCGGA-3′,

CD4 Leader:

5′-ATGAACCGGGGAGTCCCTTTTAGGCACTTGCTTCTGGTGCTGCAACT
GGCGCTCCTCCCAGCAGCCACTCAGGGA-3′,

IL-2 Leader:

5′-ATGTACAGGATGCAACTCCTGTCTTGCATTGCACTAAGTCTTGCACT
TGTCACAAACAGT-3′,

MCP Leader:

5′-TGAAAGTCTCTGCCGCCCTTCTGTGCCTGCTGCTCATAGCAGCCACC
TTCATTCCCCAAGGGCTCGCT-3′,

Short Native IL-15 Leader:

5′-ATGTCTTCATTTTGGGCTGTTTCAGTGCAGGGCTTCCTAA-3′

Long Native IL-15 Leader:

ATGAGAATTTCGAAACCACATTTGAGAAGTATTTCCATCCAGTGCTACTT
GTGTTTACTTCTAAACAGTCATTTTCTAACTGAAGCTGGCATTCATGTCT
TCATTTTGGGCTGTTTCAGTGCAGGGCTTCCTAAAACAGAAGCC

The components are functionally linked when they are connected such that the sequence(s) of the gene(s) which is/are present is/are transcribed under the influence of the transcription regulation.

The invention furthermore relates to a vector which contains at least one nucleic acid according to the invention.

Within the meaning of the present invention, vectors can be plasmids, shuttle vectors, phagemids, cosmids, adenoviral vectors, retroviral vectors, expression vectors and vectors which are effective in gene therapy.

Within the meaning of the present invention, expression vectors encompass at least one nucleic acid according to the invention, at least one translation initiation signal, a translation termination signal and/or a polyadenylation signal for expression in eukaryotes.

Commercially obtainable expression vectors, in particular for expression in mammalian cells, for example pIRES (from Clontech, Palo Alto, USA), pCI-neo vector (from Promega, Madison, USA), pCMV-Script (from Stratagene, La Jolla, USA), and pCDNA vector (from Invitrogen, Paisley, UK) are suitable for incorporating the NA according to the invention.

Vectors according to the invention which are effective in gene therapy are, for example, viral vectors, for example adenoviral vectors, retroviral vectors or vectors which are based on RNA virus replicons (Lindemann et al., 1997, Mol. Med. 3: 466-76; Springer et al., 1998, Mol. Cell. 2: 549-58; Khromykh, 2000, Curr. Opin. Mol. Ther.; 2: 555-69).

Vectors which are effective in gene therapy can also be obtained by complexing the nucleic acid fragments according to the invention with liposomes. In the lipofection, small unilamellar vesicles composed of cationic lipids are prepared by ultrasonicating the liposome suspension. The DNA is bound ionically on the surface of the liposomes, specifically in a ratio which is such that a positive net charge remains and 100% of the plasmid DNA is complexed by the liposomes. In addition to the DOTMA (1,2-dioleyloxypropyl-3-trimethylammonium bromide) and DPOE (dioleoxylphosphatidylethanolamine) lipid mixtures, a large number of new lipid formulations have by now been synthesized and tested for their transfection efficiency in a variety of cell lines (Behr et al. 1989, Proc. Natl. Acad. Sci. USA 86: 6982-6986; Gao and Huang, 1991, Biochem. Biophys. Acta 1189, 195-203; Felgner et al. 1994, J. Biol. Chem. 269, 2550-2561). Examples of the new lipid formulations are DOTAP N-[1-(2,3-dioleoyloxy)propyl]-N,N,N-trimethylammoniumethyl sulfate or DOGS (TRANSFECTAM; dioctadecylamidoglycylspermine). Auxiliary substances which increase the transport of nucleic acids into the cells can, for example, be proteins or peptides which are bound to DNA or synthetic peptide-DNA molecules which enable the nucleic acid to be transported into the cell nucleus (Schwartz et al., 1999, Gene Therapy 6: 282; Branden et al. 1999, Nature Biotechs. 17: 784). Auxiliary substances also encompass molecules which enable nucleic acids to be released into the cell cytoplasm (Planck et al., 1994, J. Biol. Chem. 269, 12918; Kichler et al., 1997, Bioconj. Chem. 8, 213) or, for example, liposomes (Uhlmann and Peimann, 1990, Chem. Rev. 90, 544).

Part of the subject matter of the invention is a cell which contains at least one nucleic acid according to the invention and/or at least one vector according to the invention.

This cell is preferably a precursor cell, an immortalized cell or a stem cell, in particular a pluripotent or multipotent embryonic, fetal, neonatal or adult stem cell. Such pluripotent embryonic stem cells or cell lines can be isolated from the inner cell mass of blastocytes (Robertson, Embryo-derived stem cell lines, in Teratocarcinomas and embryonic stem cells: a practical approach, Robertson, editor, IRL Press, Washington D.C., 1987). Particularly preferred stem cells, which originate from adult tissue, comprise, for example, neuronal stem cells, stem cells from the bone marrow, mesenchymal stem cells, hematopoietic stem cells, epithelial stem cells, stem cells from the digestive tract and duct stem cells.

Examples of cells according to the invention are epithelial cells, vascular cells, liver cells, heart cells, skin cells, muscle cells, nerve cells, bone marrow cells, CHO cells (ovary cells) and cells from the pancreatic gland, from the kidney, from the eye or from the lung.

The cell according to the invention is, in particular, a mammalian cell, including a human cell. This cell can originate, for example, from a human, a mouse, a rat, a guinea pig, a rabbit, a cow, a goat, a sheep, a horse, a pig, a dog, a cat or a monkey, preferably from a human.

The cells according to the invention can also be used for expressing a heterologous gene.

The cell according to the invention is preferably present in the form of a cell line. A cell line according to the invention can be prepared by transfecting, transforming or infecting a cell line with a nucleic acid according to the invention or a vector according to the invention using methods with which the skilled person is familiar, for example transfection, transformation, electroporation, microinjection or infection.

Another part of the subject matter of the invention is a pharmaceutical which comprises at least one fusion protein according to the invention, at least one nucleic acid according to the invention, at least one vector according to the invention and/or at least one cell according to the invention and, where appropriate, suitable auxiliary substances and/or additives.

Suitable auxiliary substances and/or additives, which are used, for example, to stabilize or preserve the pharmaceutical or diagnostic agent, are well known to the skilled person. Examples of these auxiliary substances and/or additives are physiological sodium chloride solutions, Ringer glucose, glucose, Ringer lactate, demineralized water, stabilizers, antioxidants, complexing agents, antimicrobial compounds, proteinase inhibitors and/or inert gases.

The pharmaceutical according to the invention can, for example, be used for the prophylaxis, therapy or diagnosis of diseases. These diseases include, for example:
rheumatic diseases, for example rheumatoid arthritis, Sjögren's syndrome, scleroderma, dermatomyositis, polymyositis, Reiter's syndrome or Behcet's disease,
type I or type II diabetes,
autoimmune diseases of the thyroid gland, for example Basedow's disease disease, Hashimoto's thyroiditis,
autoimmune diseases of the central nervous system, for example multiple sclerosis,
skin diseases, for example psoriasis, neurodermitis,
inflammatory intestinal diseases, for example Crohn's disease,
immunodeficiency diseases, for example AIDS,
vascular diseases,
transplantation sequelae, for example transplant rejection reactions, and
tumor diseases.

The pharmaceutical according to the invention is administered using methods with which the skilled person is familiar, for example intravenously, intraperitoneally, intramuscularly, subcutaneously, intracranially, intraorbitally, by the intracapsule route, intraspinally, transmuscularly, topically or orally. Other methods of administration are, for example, systemic or local injection, perfusion or catheter-based administration.

The pharmaceutical according to the invention can, for example, be administered in oral administration forms such as tablets or capsules, by way of the mucous membrane, e.g. the nose or the oral cavity, in the form of sprays into the lung or in the form of dispositories implanted under the skin. Transdermal therapeutic systems (TTSs) are disclosed, for example, in EP 0 944 398-A1, EP 0 916 336-A1, EP 0 889 723-A1 or EP 0 852 493-A1.

The pharmaceutical can be introduced into the organism either using an ex vivo approach, in which the cells are removed from the patient, genetically modified, for example by means of DNA transfection, and, after that, introduced into the patient once again, or using an in vivo approach, in which vectors according to the invention which are effective in gene therapy are introduced into the body of the patient as naked DNA or using viral or nonviral vectors according to the invention or cells according to the invention.

It is known in the prior art that the dosing of pharmaceuticals depends on several factors, for example on the bodyweight, on the general state of health, on the extent of the body surface, on the age of the patient and on interaction with other medicaments. A dose also depends on the type of the administration. The dose therefore has to be determined by the skilled person for each patient on an individual basis. The pharmaceutical can be administered once or several times a day and be administered over a period of several days; this can also be determined by the skilled person.

Another part of the subject matter of the invention relates to a human or animal organospecific tissue and/or to a human or animal mammalian organ which contains at least one fusion protein, at least one nucleic acid encoding said fusion protein, at least one vector containing at least one said nucleic acid and/or at least one cell containing at least one said nucleic acid and/or at least one said vector, with the fusion protein containing a wild-type IL-15 and an Fc fragment.

The fusion protein of the human or animal organospecific tissue according to the invention and/or of the human or animal mammalian organ according to the invention preferably contains a wild-type IL-15, on the one hand, and, on the other hand, a human or murine IgG1, a human IgG2, a murine IgG2a, a murine IgG2b, a human or murine IgG3 or a human IgG4, preferably a human IgG1 or a murine IgG2a, in particular an IgG1, particularly preferably not a murine IgG2b.

Human or animal organospecific tissue of the present invention can, for example, be tissue from the pancreatic gland, including, for example, the Langerhans islet cells, and also heart, heart muscle, kidney, liver, lung, spleen, cartilage, ligament, retina, cornea, bone marrow, skin, nerve and/or muscle tissue.

Human or animal mammalian organs of the present invention can, for example, be the pancreatic gland, the heart, the pancreatic gland, the kidney, the liver, the lung, the spleen, the eye and/or the skin.

Another part of the subject matter of the invention is a transgenic nonhuman mammal which at least one fusion protein, at least one nucleic acid which encodes said fusion protein, at least one vector which contains at least one said nucleic acid and/or at least one cell which contains at least one said nucleic acid and/or at least one said vector, with the fusion protein containing a wild-type IL-15 and an Fc fragment.

The fusion protein of the transgenic nonhuman mammal according to the invention preferably contains a wild-type IL-15, on the one hand, and, on the other hand, a human or murine IgG1, a human IgG2, a murine IgG2a, a murine IgG2b, a human or murine IgG3 or a human IgG4, preferably a human IgG1 or a murine IgG2a, in particular an IgG1, particularly preferably not a murine IgG2b.

In general, transgenic animals exhibit an expression of nucleic acids which is tissue-specifically increased and are, therefore, very suitable for analyzing immune reactions, for example. Preference is given to using transgenic mice.

An example of a nonhuman mammal according to the invention is a mouse, a rat, a guinea pig, a rabbit, a cow, a goat, a sheep, a horse, a pig, a dog, a cat or a monkey.

Other parts of the subject matter of the invention are also the uses of a fusion protein, of a nucleic acid encoding said fusion protein, of a vector containing at least one said nucleic acid and/or of a cell containing either at least one said nucleic acid and/or one said vector containing at least one said nucleic acid, with the fusion protein containing a wild-type IL-15 and an Fc fragment, or of a human or animal organospecific tissue according to the invention and/or of a human or animal mammalian organ according to the invention:
for inhibiting an IL-15-mediated cellular event,
for inhibiting the interaction of an IL-15 with its receptor and/or
for the prophylaxis and/or therapy of transplantation sequelae, in particular transplantation rejection reactions, and/or autoimmune diseases.

Another part of the subject matter of the invention is the use of a fusion protein, of a nucleic acid encoding said fusion protein, of a vector containing at least one said nucleic acid and/or of a cell containing at least one said nucleic acid and/or one said vector, with the fusion protein containing a wild-type IL-15 and an Fc fragment, for lysing cells which are expressing an IL-15 receptor.

The fusion protein of the uses according to the invention preferably contains a wild-type IL-15, on the one hand, and, on the other hand, a human or murine IgG1, a human IgG2, a murine IgG2a, a murine IgG2b, a human or murine IgG3 or a human IgG4, preferably a human IgG1 or a murine IgG2a, in particular an IgG1, particularly preferably not a murine IgG2b.

The uses according to the invention are preferably effected in or in connection with a human or animal mammal. Within the meaning of the present invention, a human mammal is to be understood as being a human while, within the meaning of the present invention, an animal mammal is to be understood, for example, as being a mouse, a rat, a guinea pig, a rabbit, a cow, a goat, a sheep, a horse, a pig, a dog, a cat or a monkey.

Another part of the subject matter of the present invention is the use of the human or animal organospecific tissue according to the invention and/or of the human or animal mammalian organ according to the invention for transplantation into a human or animal mammal. The transplantation is preferably an autotransplantation, an allotransplantation or a xenotransplantation.

Transplantation is the transfer of living material, e.g. of cells, tissues or organs, from one part of the body to another (autogenic transplantation) or from one individual to another (allogenic, syngenic and xenogenic transplantation) (Klein, J. S, (1991) Immunologie [Immunology], 1st edition, VHC Verlagsgesellschaft, Weinheim, p. 483) using methods which are well known to the skilled person. In connection with transplantation into another organism, a distinction is made between
synotransplantation, in which donor and recipient belong to the same species and are completely, or to a large extent, genetically identical,
allotransplantation, in which the donor and recipient belong to the same species but are immunogenetically different, and
xenotransplantation, in which the donor and recipient do not belong to the same species and are consequently completely different immunogenetically.

A process for preparing a fusion protein according to the invention, which process contains the following steps:
a. Introducing at least one nucleic acid according to the invention and/or at least one vector according to the invention into a cell, and
b. expressing the nucleic acid under suitable conditions,
   is also an aspect of the invention.

Methods for introducing nucleic acids, vectors and genes, for example differentiation marker genes or transfection marker genes, into cells are well known to the skilled person and encompass the methods which are customary in the prior art, for example electroporation, injection, transfection and/or transformation. These methods are particularly preferred when the substance comprises naked nucleic acids, in particular DNA.

Suitable conditions for expressing the nucleic acid can, for example, be created by means of expression vectors, for example by means of the abovementioned expression vectors and regulatable elements, for example promotors or regulatory nucleic acid sequences. In general, expression vectors also contain promotors which are suitable for the given cell or for the gene which is in each case to be transcribed.

Examples of regulatable elements which permit constitutive expression in eukaryotes are promotors which are recognized by RNA polymerase II. Examples of these promotors for constitutive expression in all cell and tissue types are the CD11c promotor, the pGk (phosphoglycerate kinase) promotor, the CMV (cytomegalovirus) promotor, the TK (thymidine kinase) promotor, the EF1α (elongation factor 1 alpha) promotor, the SV40 (simian virus) promotor, the RSV (Rous sarcoma virus) promotor and the pUB (ubiquitin) promotor.

Examples of regulatable elements which permit cell-specific or tissue-specific expression in eukaryotes are promoters or activator sequences composed of promoters or enhancers of genes which encode proteins which are only expressed in certain cell types. Examples of these promoters are the insulin promotor for beta cells of the pancreas, the Sox-2 promotor for nerve cells, the albumin promotor for liver cells, the myosin heavy chain promotor for muscle cells, the VE-cadherin promotor for endothelial cells and the keratin promotor for epithelial cells.

Other examples of regulatable elements which permit regulatable expression in eukaryotes are RU486-inducible promotors and the tetracycline operator in combination with a corresponding repressor (Gossen M. et al., (1994) Curr. Opin. Biotechnol. 5, 516-20).

The expression can also be controlled by way of regulatory nucleotide sequences which influence expression quantitatively and/or in a time-dependent manner. These sequences include, for example, enhancer sequences, leader sequences, polyadenylation sequences, IRES sequences and introns.

Another part of the subject matter of the invention is an in-vitro process for preparing a human or animal organospecific tissue according to the invention and/or a human or animal mammalian organ according to the invention, which process contains the following steps:
a. Introducing, into at least one stem cell, one precursor cell and/or one immortalized cell of a human or animal organospecific tissue and/or of a human or animal mammalian organ, in the first place at least one nucleic acid encoding a fusion protein and/or at least one vector containing at least one said nucleic acid, with the fusion protein containing a wild-type IL-15 and an Fc fragment, and, in the second place, at least one suitable differentiation marker gene,
b. differentiating the cell from step a.,
c. selecting the differentiated cell from step b., and
d. introducing the selected cell from step c. into at least one human or animal organospecific tissue and/or into at least one human or animal mammalian organ.

In a preferred embodiment, at least one suitable transfection marker gene is introduced, in the above-described process according to the invention, after, before, or at the same time as, step a. and the transfected cell from step a. is preferably selected after step a.

Suitable conditions for differentiating the cells can, for example, be created by adding growth factors which initiate the desired cell differentiation.

A large number of methods for selecting cells are known to the skilled person.

In order to select the differentiated cells from other cells, the process according to the invention preferably contains a positive selection scheme. In this connection, a marker gene, for example a gene which transfers an antibiotic resistance, is introduced into the cell before, during or after the differentiation step and expressed under suitable conditions. These conditions can, for example, consist of the expression of the marker gene being under the control of a promotor which is only active in the desired cells.

Expression of the marker gene transfers a resistance to the antibiotic to the cells which have been successfully differentiated. The selection of the cells which follows the differentiation can therefore be readily effected by, for example, bringing the cells into contact with the corresponding antibiotic. Cells which do not contain the corresponding antibiotic resistance die such that only the differentiated cells survive. The bringing-into-contact within the meaning of this invention can be effected, for example, by adding the active substances to the nutrient medium of a cell culture.

An antibiotic according to the invention is understood as being an antibiotic against which the antibiotic resistance gene(s) which is/are used as selection cassette according to the invention generate(s) a resistance. After the antibiotic has been added to the cultured stem cells, the only stem cells to survive and differentiate are essentially those which harbor the reporter gene expression vector.

Preference is given to introducing a second marker gene into the cells, thereby making it possible to select the cells into which the nucleic acid and/or the vector has been successfully introduced in accordance with step a. of the process. By means of this double selection, it is possible to obtain a population of the desired cells which is approx. 90%, preferably approx. 95-100%, pure.

It is possible to use differentiation marker genes and transfection marker genes, for example, for these selections. Genes which mediate resistance to given toxic substances, for example antibiotics, are predominantly used as genes of this nature. The antibiotics which are most frequently employed in this context are neomycin, hygromycin (hph), zeocin (Sh ble) and puromycin (pacA).

Other genes which are suitable for the selection, in particular for selecting stem cells, are, for example, genes which regulate the expression of surface molecules or of fluorescence markers, e.g. GFP, and which can be used to purify, by means of cell sorting, the cells which are to be selected. Other examples are genes which encode an enzyme activity which converts a precursor of a toxic substance, i.e. what is termed a “prodrug”, into a toxic substance. In this case, the selection can be negative, i.e. the only cells to survive are those which are not expressing the promotor located upstream of the gene.

Another part of the subject matter of the invention is a process for generating a transgenic nonhuman mammal according to the invention, which process comprises the following steps:
a. Introducing, into at least one oocyte, one stem cell, one precursor cell and/or one immortalized cell of a nonhuman mammal, on the one hand at least one nucleic acid encoding a fusion protein and/or at least one vector containing at least one said nucleic acid, with the fusion protein containing a wild-type IL-15 and an Fc fragment, and, on the other hand, at least one suitable transfection marker gene,
b. selecting the transfected cell from step a.,
c. introducing the cell which has been selected in accordance with step b. into at least one nonhuman mammalian blastocyte,
d. introducing the blastocyte from step c. into a nonhuman, preferably pseudopregnant, mammalian foster mother, and
e. identifying the transgenic nonhuman mammal which has developed from said blastocyte.

The methods for introducing blastocytes are known to the skilled person. The blastocyte can, for example, be introduced by injection (Hogan, B., Beddington, R., Constantini, F. and Lacy, E., A laboratory Manual (1994), Cold Spring Harbor Laboratory Press).

A transgenic nonhuman mammal can be identified, for example, by extracting genomic DNA from the transgenic nonhuman mammal, for example from the tail of a mouse. In a subsequent PCR (polymerase chain reaction), use is made of primers which specifically recognize the transgene for the nucleic acid according to the invention. Integration of the transgene can be detected in this way.

Another possibility for effecting the identification is by means of southern blotting. In this method, genomic DNA is transferred to a membrane and detected using DNA probes, for example radioactively labeled DNA probes, which are specific for the sought-after transgene.

Methods for producing a transgenic nonhuman mammal according to the invention by means of regenerating a nonhuman stem cell, oocyte, precursor cell or immortalized cell to give a transgenic nonhuman animal, in particular transgenic mice, are known to the skilled person from DE 196 25 049 and the US patents U.S. Pat. No. 4,736,866; U.S. Pat. No. 5,625,122; U.S. Pat. No. 5,698,765; U.S. Pat. No. 5,583,278 and U.S. Pat. No. 5,750,825, and encompass transgenic animals which can be produced, for example, by directly injecting expression vectors according to the invention into embryos or spermatocytes or by transfecting expression vectors into embryonic stem cells (Polites and Pinkert: DNA Mikroinjection and Transgenic Animal Production, pages 15-68 in Pinkert, 1994: Transgenic Animal Technology: A Laboratory Handbook, Academic Press, San Diego, USA; Houdebine 1997, Harwood Academic Publishers, Amsterdam, The Netherlands; Doetschman: Gene Transfer in Embryonic Stem Cells, pages 115-146 in Pinkert, 1994, see above; Wood: Retrovirus-Mediated Gene Transfer, pages 147-176 in Pinkert, 1994, see above; Monastersky: Gene Transfer Technology: Alternative Techniques and Applications, pages 177-220 in Pinkert, 1994, see above).

A transgenic nonhuman mammal according to the invention can also be prepared by directly injecting a nucleic acid according to the invention into the pronucleus of a nonhuman mammal.

A large number of methods for preparing transgenic animals, in particular transgenic mice, are also known to the skilled person from, inter alia, WO 98/36052, WO 01/32855, DE 196 25 049, U.S. Pat. No. 4,736,866, U.S. Pat. No. 5,625,122, U.S. Pat. No. 5,698,765, U.S. Pat. No. 5,583,278 and U.S. Pat. No. 5,750,825 and encompass transgenic animals which can be produced, for example, by directly injecting vectors according to the invention into embryos or spermatocytes or by transfecting vectors or nucleic acids into embryonic stem cells (Polites and Pinkert, in Pinkert, (1994) Transgenic animal technology, A Laboratory Handbook, Academic Press, London, UK, pages 15 to 68; Doetschmann, in Pinkert, 1994, see above, pages 115 to 146).

Another part of the subject matter of the invention relates to a transgenic nonhuman mammal, and also its offspring, which have been produced in accordance with the above-described process according to the invention.

In other embodiments, the stem cell which is used in said in-vitro process according to the invention for preparing a human or animal organospecific tissue according to the invention and/or a human or animal mammalian organ according to the invention, and in the process for producing a transgenic nonhuman mammal according to the invention, is a pluripotent or multipotent embryonic, fetal, neonatal or adult stem cell.

Part of the subject matter of the invention is the use of a transgenic nonhuman according to the invention for obtaining a cell, an organospecific tissue and/or a mammalian organ for allotransplantation and/or xenotransplantation.

When the cell is transplanted, this can be effected, for example, using an implantation method or using a method for injection by catheter through the blood vessel wall.

Within the meaning of the present invention, “obtaining” is to be understood as meaning the removal of said cell, tissue and/or organ from the body of a transgenic nonhuman mammal according to the invention. Appropriate methods for performing this removal are well known to the skilled person.

The use of a transgenic nonhuman mammal according to the invention, of a human or animal organospecific tissue according to the invention and/or of a human or animal mammalian organ according to the invention for finding pharmacologically active compounds and/or identifying toxic substances is also part of the subject matter of the invention.

Such a method could consist, for example, in sowing cells of the present invention on a 96-well microtiter plate and then adding a pharmacologically active or toxic substance to be investigated and subsequently analyzing, by means of determining the cell count, whether the substance has increased the rate of cell death.

Within the meaning of the invention, the terms pharmacologically active compound and toxic substance are to be understood as meaning all those molecules, compounds and/or compositions and substance mixtures which, under suitable conditions, exert a pharmacologically or toxic influence on individual cells, individual tissues, individual organs or the whole body of an animal or human mammal. Possible pharmacologically active compounds and toxic substances can be simple chemical (organic or inorganic) molecules or compounds, nucleic acids or analogs of nucleic acids, nucleic acid antisense sequences, peptides, proteins or complexes and antibodies. Examples are organic molecules which originate from substance libraries and which are analyzed for their pharmacological or toxic activity.

Examples of pharmacologically active compounds are active compounds which exert an influence on:
the ability of cells to divide and/or survive,
the secretion of proteins, for example of insulin by beta cells of the pancreas or of dopamine by nerve cells,
the contraction of muscle cells, and/or
the migratory behavior of cells.

When being used on the whole body of an animal or human mammal, this is to be understood as meaning an influence on, for example,
the cardiovascular system,
the nervous system, and also
the metabolic activities.

Examples of toxic substances are active compounds which
after given signals, for example stress, stimulate cells to undergo apoptosis,
exert an influence on the cardiovascular system,
exert an influence on the nervous system, and/or
exert an influence on the metabolic activities.

The pharmacologically active compounds and toxic substances which have been identified can be used, where appropriate in combination or together with suitable additives and/or auxiliary substances, for producing a diagnostic agent or a pharmaceutical for the diagnosis, prophylaxis and/or therapy of transplantation sequelae and/or autoimmune diseases, as listed above by way of example.

The following figures and examples are intended to clarify the present invention without, however, restricting it.

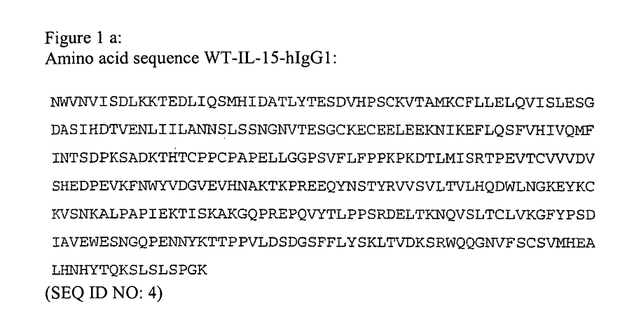

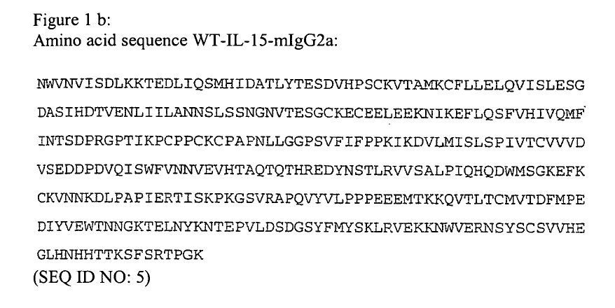

FIG. 1a depicts the amino acid sequence WT-IL-15-hIgG1,

FIG. 1b depicts the amino acid sequence WT-IL-15-mIgG2a,

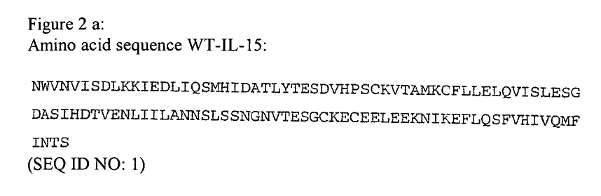

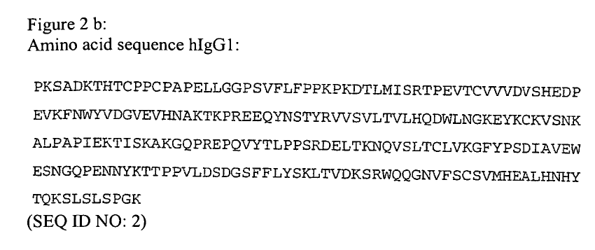

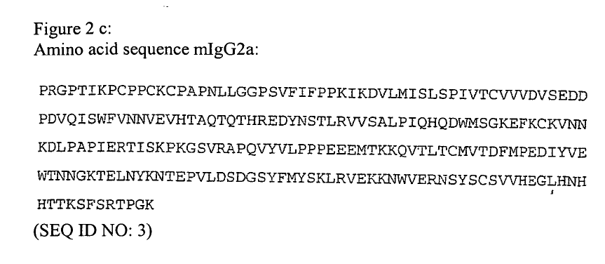

FIG. 2a depicts the amino acid sequence WT-IL-15,

FIG. 2b depicts the amino acid sequence hIgG1,

FIG. 2c depicts the amino acid sequence mIgG2a,

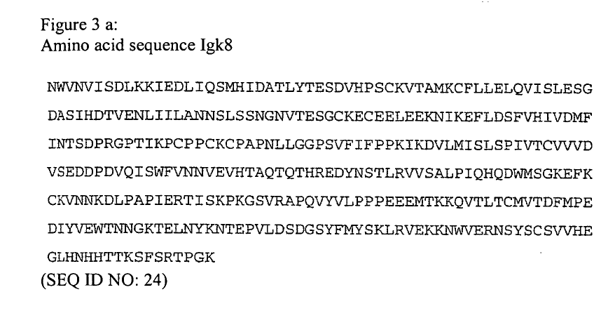

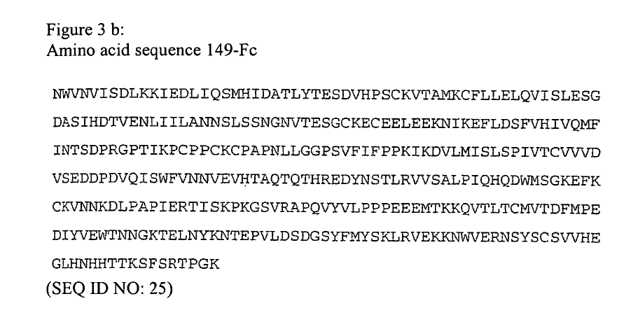

FIG. 3a depicts the amino acid sequence Igk8,

FIG. 3b depicts the amino acid sequence 149-Fc,

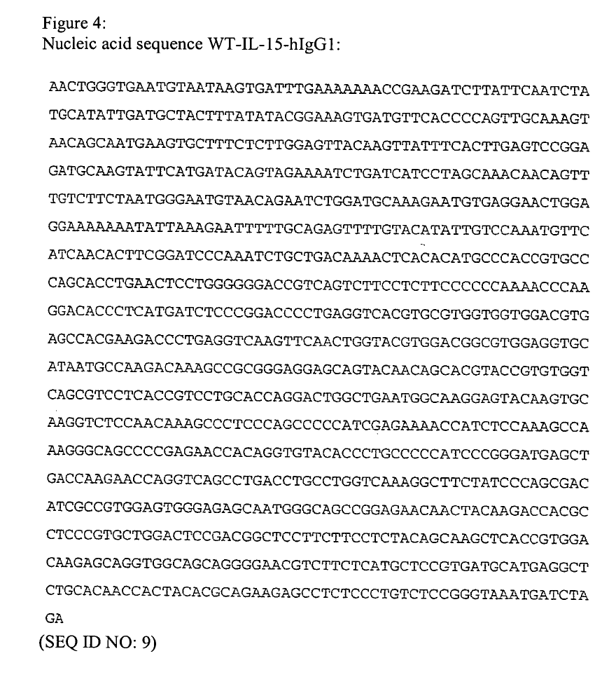

FIG. 4 depicts the nucleic acid sequence WT-IL-15-hIgG1,

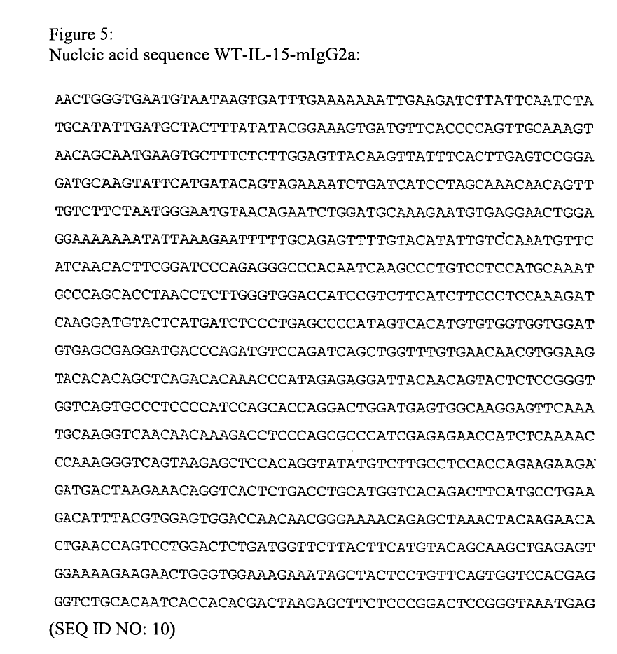

FIG. 5 depicts the nucleic acid sequence WT-IL-15-mIgG2a,

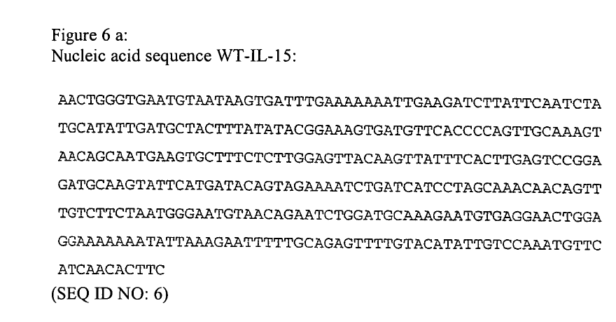

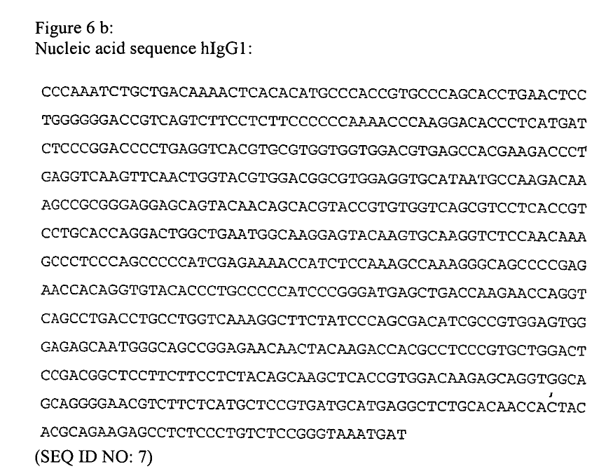

FIG. 6a depicts the nucleic acid sequence WT-IL-15,

FIG. 6b depicts the nucleic acid sequence hIgG1,

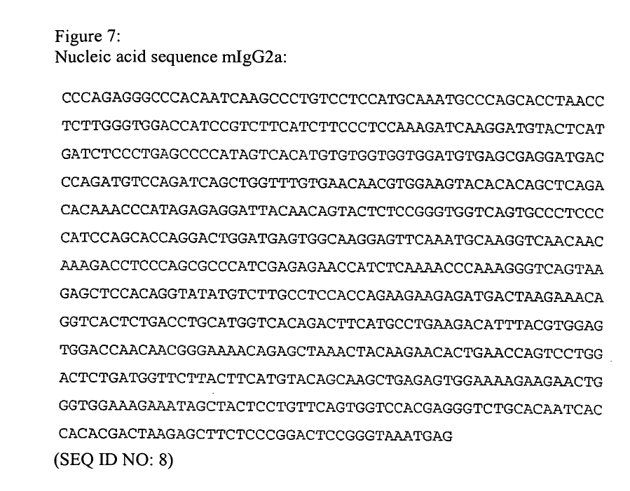

FIG. 7 depicts the nucleic acid sequence mIgG2a,

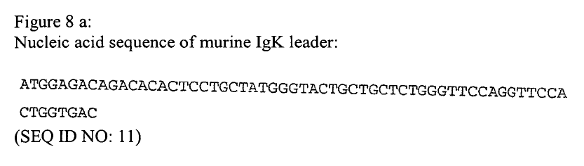

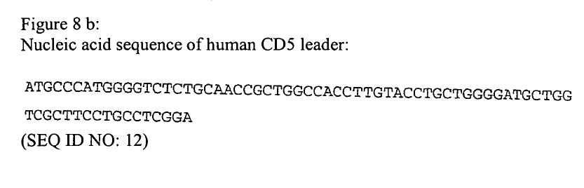

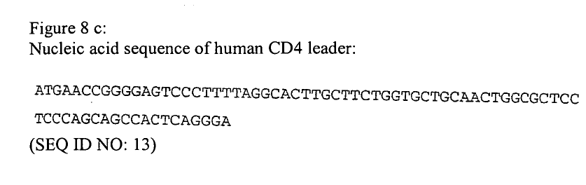

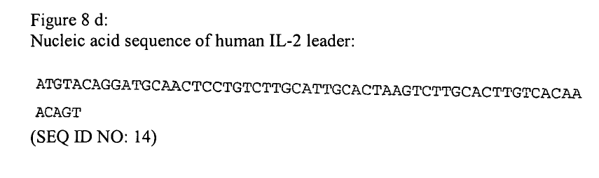

FIG. 8a depicts the nucleic acid sequence of the murine IgK leader,

FIG. 8b depicts the nucleic acid sequence of the human CD5 leader,

FIG. 8c depicts the nucleic acid sequence of the human CD4 leader,

FIG. 8d depicts the nucleic acid sequence of the human IL-2 leader,

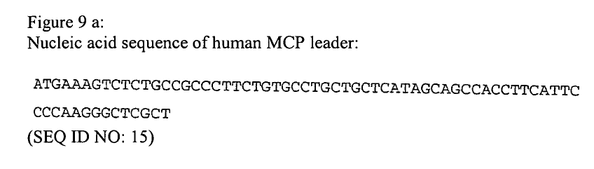

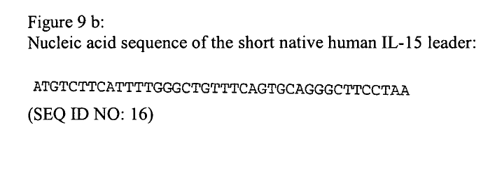

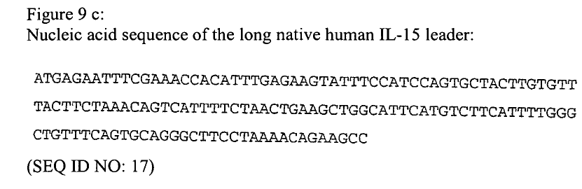

FIG. 9a depicts the nucleic acid sequence of the human MCP leader,

FIG. 9b depicts the nucleic acid sequence of the short native human IL-15 leader,

FIG. 9c depicts the nucleic acid sequence of the long native human IL-15 leader,

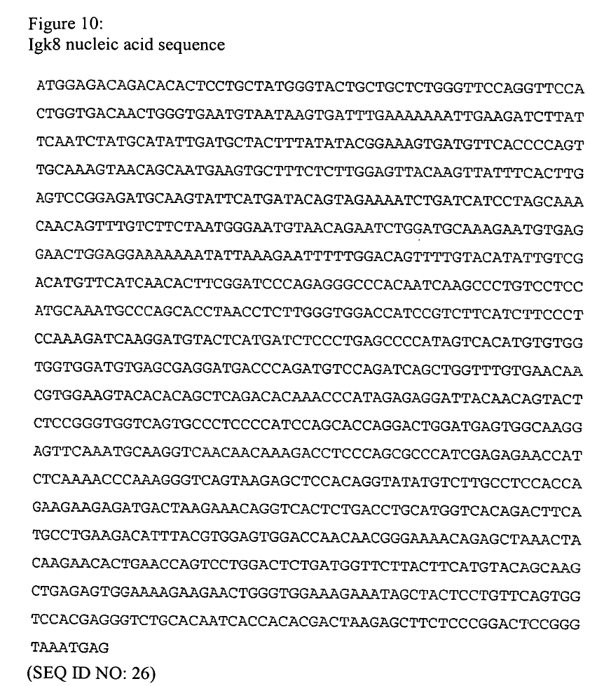

FIG. 10 depicts the nucleic acid sequence Igk8,

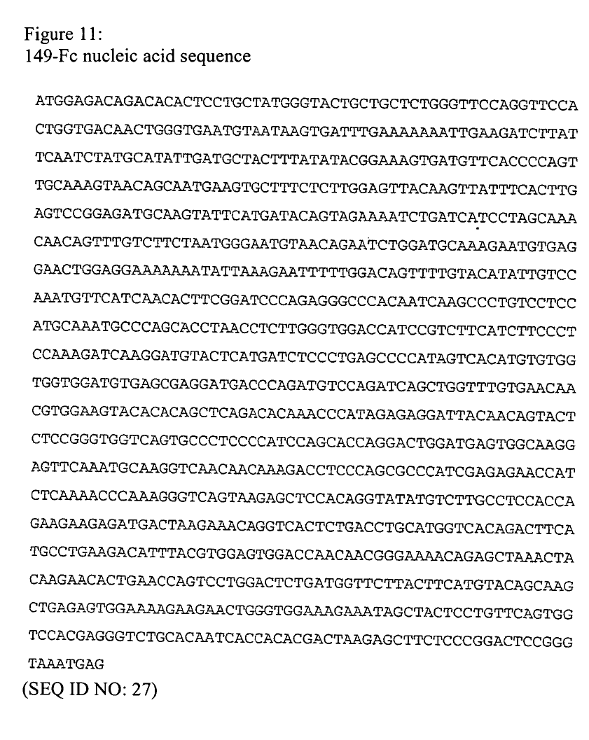

FIG. 11 depicts the nucleic acid sequence 149-Fc,

FIG. 12 depicts the inhibitory or proliferation-promoting effect of different protein constructs on the IL-15-mediated proliferation of CTLL-2 cells.

Explanation: hIgG1 stands for human IgG1 and mIgG2a stands for murine IgG2a.

Other parts of the subject matter of the present invention relate to:
(i) A fusion protein composed of a wild-type IL-15 and an IgG Fc fragment, with the exception of a murine IgG2b Fc fragment.
(ii) A fusion protein in accordance with (i), characterized in that the IgG Fc fragment is a human or murine IgG1, a human IgG2, a murine IgG2a, a human or murine IgG3 or a human IgG4.
(iii) A fusion protein according to (i) or (ii) which contains the amino acid sequence SEQ ID NO:1 or an allelic variant thereof.
(iv) A fusion protein according to (i) or (ii) which contains the amino acid sequence SEQ ID NO:2 or an allelic variant thereof.
(v) A fusion protein according to (i) or (ii) which contains the amino acid sequence SEQ ID NO:3 or an allelic variant thereof.
(vi) A fusion protein according to (i) or (ii) which contains the amino acid sequence SEQ ID NO:4 or an allelic variant thereof.
(vii) A fusion protein according to (i) or (ii) which contains the amino acid sequence SEQ ID NO:5 or an allelic variant thereof.
(viii) A nucleic acid which encodes a fusion protein according to at least one of (i) to (vii).
(ix) A nucleic acid according to (viii) which contains the DNA sequence SEQ ID NO:6 or an allelic variant thereof.
(x) A nucleic acid according to (viii) which contains the DNA sequence SEQ ID NO:7 or an allelic variant thereof.
(xi) A nucleic acid according to (viii) which contains the DNA sequence SEQ ID NO:8 or an allelic variant thereof.
(xii) A nucleic acid according to (viii) which contains the DNA sequence SEQ ID NO:9 or an allelic variant thereof.
(xiii) A nucleic acid according to (viii) which contains the DNA sequence SEQ ID NO:10 or an allelic variant thereof.
(xiv) A fusion protein which is encoded by a nucleic acid according to one of (ix)-(xiii).
(xv) A vector which contains at least one nucleic acid according to at least one of (viii)-(xiv).
(xvi) A cell which contains at least one nucleic acid according to at least one of (xiii)-(xiv) and/or at least one vector according to (xv).
(xvii) A cell according to (xvi), characterized in that the cell is a stem cell, a precursor cell and/or an immortalized cell.
(xviii) A cell according to (xvii), characterized in that the cell is a pluripotent or multipotent embryonic, fetal, neonatal or adult stem cell.
(xix) A cell according to at least one of (xvi) to (xviii) in the form of a cell line.
(xx) A pharmaceutical which comprises at least one fusion protein according to one of (i) to (vii) and (xiv), at least one nucleic acid according to one of (viii) to (xiii), at least one vector according to (xv) and/or at least one cell according to one of (xvi) to (xviii) and suitable auxiliary substances and/or additives.
(xxi) A human or animal organospecific tissue and/or human or animal mammalian organ which contains at least one fusion protein, in particular according to one of (i)-(vii) and (xiv), at least one nucleic acid which encodes said fusion protein, in particular according to one of (viii)-(xiii), at least one vector which contains at least one said nucleic acid, in particular according to (xv), and/or at least one cell, in particular according to one of (xvi)-(xviii), which contains at least one said nucleic acid and/or at least one said vector, with the fusion protein containing a wild-type IL-15 and an Fc fragment.
(xxii) A transgenic nonhuman mammal which comprises at least one fusion protein, in particular according to one of (i)-(vii) and (xiv), at least one nucleic acid encoding said fusion protein, in particular according to one of (viii)-(xiii), at least one vector, in particular according to (xv), which contains at least one said nucleic acid and/or at least one cell, in particular according to one of (xvi)-(xviii), which contains at least one said nucleic acid and/or at least one said vector, with the fusion protein containing a wild-type IL-15 and an Fc fragment.
(xxiii) The use of a fusion protein, in particular according to one of (i)-(vii) and (xiv), of a nucleic acid, in particular according to one of (viii)-(xiii), of a vector, in particular according to (xv), and/or of a cell, in particular according to one of (xvi)-(xviii), with the fusion protein containing a wild-type IL-15 and an Fc fragment, or of a human or animal organospecific tissue and/or of a human or animal mammalian organ according to (xxi) for producing a medicament for inhibiting an IL-15-mediated cellular event.
(xxiv) The use of a fusion protein, in particular according to one of (i)-(vii) and (xiv), of a nucleic acid, in particular according to one of (viii)-(xiii), of a vector, in particular according to (xv) and/or of a cell, in particular according to one of (xvi)-(xviii), with the fusion protein containing a wild-type IL-15 and an Fc fragment, or of a human or animal organospecific tissue and/or of a human or animal mammalian organ according to (xxi) for producing a medicament for inhibiting the interaction of an IL-15 with its receptor.
(xxv) The use of a fusion protein, in particular according to one of (i)-(vii) and (xiv), of a nucleic acid, in particular according to one of (viii)-(xiii), of a vector, in particular according to (xv), and/or of a cell, in particular according to one of (xvi)-(xviii), with the fusion protein containing a wild-type IL-15 and an Fc fragment, for producing a medicament for lysing cells which are expressing an IL-15 receptor.
(xxvi) The use of a fusion protein, in particular according to one of (i)-(vii) and (xiv), of a nucleic acid, in particular according to one of (viii)-(xiii), of a vector, in particular according to (xv), and/or of a cell, in particular according to one of (xvi)-(xviii), with the fusion protein containing a wild-type IL-15 and an Fc fragment, or of a human or animal organospecific tissue and/or of a human or animal mammalian organ according to (xxi) for producing a medicament for the prophylaxis and/or therapy of transplantation sequelae and/or autoimmune diseases.
(xxvii) The use of a human or animal organospecific tissue and/or human or animal mammalian organ according to (xxi) for transplantation into a human or animal mammal.
(xxviii) The use according to (xxvii), characterized in that the use is an autotransplantation, allotransplantation or xenotransplantation.
(xxix) A process for preparing a fusion protein according to at least one of (i) to (vii) and (xiv), comprising the following steps:
a. introducing at least one nucleic acid according to one of (viii) to (xiii) and/or at least one vector according to (xv) into a cell, and
b. expressing the nucleic acid under suitable conditions.
(xxx) An in-vitro process for preparing a human or animal organospecific tissue and/or human or animal mammalian organ according to (xxi), comprising the following steps:
a. introducing, into at least one stem cell, one precursor cell and/or one immortalized cell of a human or animal organospecific tissue and/or of a human or animal mammalian organ, in the first place at least one nucleic acid encoding a fusion protein, with the fusion protein containing a wild-type IL-15 and an Fc fragment, and/or at least one vector containing at least one said nucleic acid, in particular according to one of (viii)-(xiii), and, in the second place, at least one suitable differentiation marker gene,
b. differentiating the cell from step a.,
c. selecting the differentiated cell from step b., and
d. introducing the selected cell from step c. into a human or animal organospecific tissue and/or into a human or animal mammalian organ.
(xxxi) The process according to (xxx), characterized in that at least one suitable transfection marker gene is introduced after, before or at the same time as, step a. and the transfected cell from step a. is preferably selected after step a.
(xxxii) The process according to one of (xxx) or (xxxi), characterized in that the cell is a pluripotent or multipotent embryonic, fetal, neonatal or adult stem cell.
(xxxiii) A process for producing transgenic nonhuman mammals according to (xxii), comprising the following steps:
a. introducing, into at least one oocyte, one stem cell, one precursor cell and/or one immortalized cell of a nonhuman mammal, on the one hand at least one nucleic acid, in particular according to one of (vii)-(xiii), encoding a fusion protein, and/or at least one vector, in particular according to (xv), containing at least one said nucleic acid, with the fusion protein containing a wild-type IL-15 and an Fc fragment, and, on the other hand, at least one suitable transfection marker gene,
b. selecting the transfected cell from step a.,
c. introducing the cell which has been selected according to step b. into at least one nonhuman mammalian blastocyte,
d. introducing the blastocyte from step c. into a nonhuman mammalian foster mother, and
e. identifying the transgenic nonhuman mammal which has developed from said blastocyte.
(xxxiv) The process according to (xxxiii), characterized in that the cell is a pluripotent or multipotent embryonic, fetal, neonatal or adult stem cell.
(xxxv) A transgenic nonhuman mammal, characterized in that it was produced using the process according to one of (xxxiii) and (xxxiv).
(xxxvi) A transgenic nonhuman mammal, characterized in that it is an offspring progeny of the mammal according to (xxxv).
(xxxvii) The use of a transgenic nonhuman mammal according to at least one of (xxii), (xxxv) and (xxxvi) for obtaining a cell, an organospecific tissue and/or a mammalian organ for allotransplantation and/or xenotransplantation.
(xxxviii) The use of a transgenic nonhuman mammal according to one of (xxii), (xxxv) and (xxxvi), of a human or animal organospecific tissue and/or of a human or animal mammalian organ according to (xxi) for finding pharmacologically active compounds and/or identifying toxic substances.

### EXAMPLES

Reagents

Unless otherwise noted, reagents such as cell culture media, enzymes, etc., were obtained from Invitrogen (previously Gibco BRL/Life Technologies), Paisley, UK, while laboratory chemicals were obtained from Roth (Karlsruhe, Germany).

### Example 1

### Replacing the Signal Sequence

The procedure started with a plasmid which contained, in the vector pSecTagA (Invitrogen, Paisley, UK), the cDNA for a fusion protein which was composed of a mutated human IL-15 and a murine IgG2a Fc moiety (hinge-C2-C3, Kim et al. 1998, see above). The IL-15 was fused to the Fc moiety by way of a BamHI cleavage site, resulting in an additional amino acid (aspartate) being inserted at the junction.

Two glutamine residues had been mutated to aspartate at positions 149 and 156 (corresponding to positions 101 and 108 after elimination of the signal sequence) in the IL-15 in order to enable the protein to bind to the alpha subunit of the IL-15 receptor but to prevent signal transduction by way of the beta and gamma subunits. The native signal sequence, which is not particularly efficient, had been removed from the human IL-15 and correspondingly the truncated cDNA had been cloned into the pSecTagA vector by way of the HindIII and XbaI cleavage sites such that the Ig kappa leader present in the plasmid was able to be used as the secretion signal. As a result of the cloning, 10 additional amino acids were located between the Ig kappa leader, present in the plasmid, and the beginning of the IL-15 sequence. In order to remove these amino acids and, if possible, improve the secretion of the protein, the Ig kappa leader was replaced with signal sequences from a variety of other proteins. In this connection, the leader sequences from human IL-2, MCP-1, CD4 and CD5 can be cloned in as an alternative to the original Ig kappa leader from which only the additional amino acids have been removed.

### Example 2

### Preparing the pSecTagA Plasmid

Since the signal sequence was to be cloned by way of a unique NheI cleavage site, which was located in the 5′ direction from the ATG start codon of the leader sequence, and a BglII cleavage site which was located in the 5′ region of the IL-15 sequence, an additional BglII cleavage site was first of all removed from vector pSecTagA. For this, vector pSecTagA without any insert was cut with BglII (mixture: 9 μg of DNA, 4 μl of 10× buffer 2, 26 μl of water and 4 μl of BglII (40 units) in a total of 40 μl, incubation at 37° C. for 2 h).

The DNA was purified from enzyme and buffer through a Pharmacia S400 Microspin column (Amersham-Pharmacia, Freiburg). 5 μl of 10× PCR buffer (Taq-Core kit, Qiagen, Hilden), 2 μl of dNTPs (10 mM each, Taq-Core kit, Qiagen), 2 μl of water and 1 μl (4 units) of DNA polymerase I (Klenow fragment) were added to 40 μl of the mixture and the whole was incubated at 37° C. for 1 h in order to fill in the BglII cleavage site. The plasmid was then loaded onto a 1% agarose gel and the band was eluted from the gel using the Concert Rapid-Gel extraction system. The entire mixture was taken up in 100 μl of water. 7.5 μl of this latter mixture were ligated, at room temperature for 1 h, together with 7.5 μl of water, 4 μl of 5× T4 ligase buffer and 1 μl of T4 ligase (1U). Half of the ligation mixture was transformed into E. coli XL1 Blue in accordance with the manufacturer's (Stratagene, La Jolla, USA) instructions.

The entire insert from the abovementioned plasmid, i.e. Ig kappa leader+10 additional amino acids-mutIL-15-mIgG2a, were once again cloned into the resulting plasmid by way of the NheI and XbaI cleavage sites. The original Ig kappa leader+10 amino acids+5′-IL-15 moiety were then removed by way of an NheI/BglII cleavage and replaced, by means of oligonucleotide cloning, with the abovementioned signal sequences.

### Example 3

### Cloning the Ig Kappa Leader

The fragment was as follows: 5′-NheI-leader-IL-15-3′, with a BglII cleavage site in the 5′ segment of the IL-15. Since this fragment was too long to be covered by a single oligonucleotide, two overlapping oligos and their complementary strands (4 oligonucleotides in all) were obtained from MWG-Biotech (Ebersberg) (sequences of the oligonucleotides, see below). The single-stranded oligonucleotides were selected such that overhanging ends for cloning into the corresponding restriction cleavage sites (NheI and BglII) were already present. The oligonucleotides were first of all phosphorylated. For this, 10 μg of each oligo was incubated, at 37° C. for 1 h, in a 20 μl mixture containing 2 μl of 10× forward buffer and 1 μl of T4 polynucleotide kinase (10 U). Equimolar quantities of in each case the strand oligo and the counterstrand oligo were then annealed by heating to 95° C. and slowly cooling down to room temperature. Before being cloned into the vector, the double-stranded oligonucleotides were ligated overnight. In each case 5 μl of the 5′ and 3′ double stranded oligos+4 μl of 5× T4 ligase buffer+5 μl of water+1 μl of T4 ligase (1 U) were incubated overnight at 4° C. The ligation mixture was then separated on a 2% agarose gel and oligodimers were eluted from the gel using the Concert Rapid Gel Extraction System and taken up in a final volume of 40 μl. The oligodimers were then used for the cloning: the ligation was carried out overnight at 12° C. (10 μl of oligodimer, 4 μl of 5× T4 ligase buffer, 4 μl of water, 1 μl of NheI/BglII-cut plasmid, 1 μl of T4 ligase (1 U)). 5 μl of a 20 μl ligation mixture were used to transform E. coli-XL10-Gold (Stratagene, in accordance with the manufacturer's instructions).

Sequences of the Ig-kappa Oligonucleotides:

5′-Ig-kappa fwd
ctagccaccatggagacagacacactcctgctatgggtactgctgctctg
ggttccaggttccactggtgacaa

complementary Ig-kappa rev:

ccagttgtcaccagtggaacctggaacccagagcagcagtacccatagca
ggagtgtgtctgtctccatggtgg

pos second forward oligo: 3′-IL-15 fwd1.1:

ctgggtgaatgtaataagtgatttgaaaaaaattga

complementary IL-15 rev1.1

gatcttcaatttttttcaaatcacttattacattcac

After annealing and ligation, the following fragment is obtained:

5′-Nhel-Ig-kappa-leader-Il-15-BglII-3′ having the sequence (double-stranded)

5′-CTAGCCACCATGGAGACAGACACACTCCTGCTATGGGTACTGCTGCT
CTGGGTTCCAGGTTCCACTGGTGACAACTGGGTGAATGTAATAAGTGATT
TGAAAAAAATTGAA-3′

complementary:

3′-GGTGGTACCTCTGTCTGTGTGAGGAGCATACCCATGACGACGAGACC
CAAGGTCCAAGGTGACCACTGAAGACCCACTTACATTATTCACTAAACTT
TTTTTAACTTCTAG-5′

Explanation:
italics+underlined: NhelII and BglII cleavage sites, respectively; bold letters: Ig-kappa leader.

The restriction patterns of the resulting clones were examined in a miniprep (QIAamp DNA Mini Kit, Qiagen, Hilden). For this, a three-fold digestion was carried out using NheI/BglII (restriction enzymes which directly excise the inserted leader) and XbaI (cuts 3′ of the Fc moiety).

The positive DNA clones were isolated, using the Qiagen Endofree Maxi kit in accordance with the manufacturer's instructions, and sequenced at GATC (Constance). The plasmid which was obtained in this way (mutIL-15 101/108)-mIgG2a with a cleaned-up Ig-kappa leader) was designated Igk8.

The procedure for the other leaders was precisely the same as that for the described Ig-kappa construct.

### Example 4

### Preparing the Constructs: WT-Fc and 149-Fc

Starting with the above-described plasmid Igk8, the individual mutants were prepared by means of PCR using a forward primer having a BglII cleavage site at the 5′ end (IL-15fw3.1: 5′-attgaagatcttattcaatctatgc-3 )

and a corresponding 3′ reverse primer (WT: 5′-ggatccgaagtgttgatgaacatttggacaatatgtacaaaactctgcaaaaattc-3′).

(149: 5′-gggatcc-gaagtgttgatgaacatttgga-3′)

10 ng of mutIL-15(101, 108)-murine Fc plasmid were used, per 25 μl mixture, as the template for the PCR reaction, with the mixture also containing in each case 25 pmoles of primer, 0.5 μl of dNTPs (Taq-Core kit, Qiagen) and 2.5 μl of 10× PCR buffer and 0.9 U of Taq polymerase (Expand High-Fidelity system, Roche, Mannheim). The DNA was amplified in 30 cycles under the conditions: 45 seconds of denaturation at 95° C., 60 seconds of annealing at 60° C. and 45 seconds of synthesis at 72° C., after which the amplificate was purified on an agarose gel with the PCR bands being eluted from the gel and taken up in 50 μl of TE buffer. 25 μl of the mixture were treated with 3 μl of 10× buffer 3 and in each case 15 U of BamHI and BglII and incubated at 37° C. for 1 hour. The DNA was purified through a Pharmacia Microspin S400 column. The IL-15 moiety, containing a double mutation, was excised from plasmid Igk8, likewise by means of a double BglII/BamHI digestion, and replaced with the IL-15 moiety containing a single mutation or the wild-type sequence. The identities of the plasmids were verified by sequencing.

### Example 5

### Preparing Protein

The proteins of the individual mutants were prepared by transiently transfecting HEK293 cells (ATCC, Manassas, USA): for this, 60 μl of Lipofectamine2000 were diluted in 2 ml of Optimem 1 medium, and 30 μg of plasmid DNA (IgK8, WT-Fc and 149-Fc) were likewise diluted in 2 ml of Optimem 1 medium, per 150 cm² plate. The two solutions were mixed and incubated at room temperature for 30 min. The DNA/liposome mixture was then added to the cell culture medium (Dulbecco's MEM+Glutamax+10%FCS+1% Pen/Strep) on 150 cm² HEK-293 plates which were approx. 80% confluent. After one day, the medium was replaced with Ultraculture medium (Biowhittaker, Verviers, Belgium) and the cell culture medium was then left on the cells for 4 days. The cell culture supernatant was collected and passed through a fluted filter (Schleicher and Schüll, Dassel) in order to remove the coarse cell constituents; it was then sterilized by filtration through a 2 μm bottle-top filter (Nalgene-Nunc, Wiesbaden) and the IL-15-Fc fusion protein was isolated by means of purification through protein A-Sepharose. For this, 0.4 ml of protein A-Sepharose which had been swollen in washing buffer (20 mM Tris/HCl, pH 8.5, 130 mM NaCl) (Amersham-Pharmacia, 50% v/v in washing buffer) was added per liter of cell culture supernatant and the mixture was shaken overnight at 4° C. in an overhead shaker. The protein A-Sepharose was collected in an empty chromatographic column and washed with at least 150 ml of washing buffer. The protein was eluted from the column in 1 ml fractions using 0.1 M glycine, pH 2.5, and immediately neutralized with 60 μl of 1 M Tris/HCl, pH 9.5. The protein was dialyzed against PBS buffer and sterilized by filtration. The concentration of the protein was determined in a BCA assay (Pierce, Rockford, USA) and its purity and identity were examined using a silver gel and western blotting (first antibody: monoclonal mouse anti-human IL-15, BD Biosciences Pharmingen, San Diego USA; second antibody: POD-goat anti-mouse, Dianova, Hamburg). The functional ability of the protein was then investigated in a proliferation assay.

### Example 6

### Proliferation Assay

CTLL-2 cells (ATCC) are murine cytotoxic T cells whose proliferation depends on IL-15 or IL-2 and which can therefore be used as indicators of the proliferation-inhibiting effect of antagonistic proteins. The cells were cultured in a medium consisting of RPMI1640 medium+10% heat-inactivated fetal calf serum (FCS)+1% Pen/Strep +20% rat T-stim with ConA (Becton Dickinson Labware, Bedford, USA), a mixture of various growth factors.

For preparing a proliferation assay, the cells were freed from residual growth factors, which were required for culturing the cells, by washing them twice with cell culture medium (RPMI 1640+10% FCS+1%Pen/Strep) and then taking them up in this medium. For this, the cells were centrifuged at 349 g for 5 min, after which the supernatant was discarded and the pellet was taken up once again in cell culture medium. The centrifugation step was repeated.

The assay took place in flat-bottomed 96-well plates and 150 μl of medium, containing 3×10⁴ cells/well, were used per well. For the negative control, the cells were only given medium containing 10% FCS without any additional factors. The positive control additionally contained recombinant human IL-15 (R&D Systems, Minneapolis, USA) at a concentration (e.g. 12.5 pg/well) which permitted half-maximal proliferation of the cells. In each case 6 negative and positive controls were set up.

In order to determine the proliferation-inhibiting effect of the abovementioned novel IL-15-Fc variants, the cells were treated with recombinant IL-15 as described for the positive control and were additionally given purified protein in the form of the 101/108 double mutant, originating from Igk8, of the wild-type protein (WT-Fc) or of the single mutant (149-Fc). In this connection, the highest concentration which was used per well was 2 μg, with dilutions, which were in each case 1:2 (1 μg, 0.5 μg, 0.25 μg, 0.125 μg, etc.), also being used. As controls, the following related proteins were used at the same concentrations: mIgG2a (BD Biosciences Pharmingen, San Diego, USA) was used as a nonspecific antibody, while use was also made of IL-2-Fc, which contains an unmutated cytokine moiety and consequently stimulates proliferation of the cells, as well as CTLA4-Fc, which is also a structurally related fusion protein but which should not have any effect on proliferation. The latter two proteins were obtained from Chimerigen (Allston, USA). All the mixtures were set up in triplicates.

The cells were incubated at 37° C. for 44±2 hours in a CO₂ incubator after which proliferation was determined using an XTT Cell Proliferation kit (Roche) in accordance with the manufacturer's instructions. For this, the two components of the kit were mixed in a ratio of 1:50 (i.e., 75 μl of XTT labeling reagent+1.5 μl of electron coupling reagent). 75 μl of the mixture were added per well and, after a 4-hour incubation at 37° C. in a CO₂ incubator, the plate was measured in an ELISA reader at 490 against 690 nm.

The result is shown in FIG. 23:

WT-Fc, 149-Fc and protein from the double mutant 101/108 (plasmid Igk8) exhibit an inhibitory effect on the IL-15-mediated proliferation of CTLL-2 cells. If anything, IL-2-Fc and IgG2a exhibit a proliferation-promoting effect.

Neg: the cells were cultured without recombinant human IL-15.

Pos: the cells were given 12.5 pg of recombinant human IL-15/well.

All the cells in the other mixtures were given 12.5 pg of recombinant human IL-15/well+the given protein at the following concentrations (from left to right): 2 μg, 1 μg, 0.5 μg, 0.25 μg, 0.125 μg and 0.0625 μg. CTLA4-Fc did not have any effect; all the values were in the positive control range (data not shown).

1-38. (canceled)

39. A fusion protein composed of a wild-type IL-15 and an IgG Fc fragment, with the exception of a murine IgG2b Fc fragment.

40. A fusion protein as claimed in claim 39, characterized in that the IgG Fc fragment is a human or murine IgG1, a human IgG2, a murine IgG2a, a human or murine IgG3 or a human IgG4.

41. A fusion protein as claimed in claim 39 which contains an amino acid sequence selected from the group consisting of SEQ ID NO:1, SEQ ID NO:2, SEQ ID NO:3, SEQ ID NO:4, and SEQ ID NO:5 or an allelic variant thereof.

42. A nucleic acid which encodes a fusion protein as claimed in claim 39.

43. A nucleic acid as claimed in claim 42 which contains a DNA sequence selected from the group consisting of SEQ ID NO:6, SEQ ID NO:7, SEQ ID NO:8, SEQ ID NO:9, and SEQ ID NO:10 or an allelic variant thereof.

44. A cell which contains at least one nucleic acid as claimed in claim 42.

45. A pharmaceutical which comprises at least one fusion protein as claimed in claim 39, or at least one nucleic acid as claimed in claim 42, and suitable auxiliary substances and/or additives.

46. Method of preventing and/or treating transplantation sequelae and/or autoimmune diseases, wherein a fusion protein as claimed in claim 39 or a nucleic acid as claimed in claim 42 is administered to a subject.

47. A process for preparing a fusion protein as claimed in claim 39, comprising the following steps:
a. Introducing at least one nucleic acid as claimed in claim 42 and/or at least one vector containing at least one nucleic acid as claimed in claim 42 into a cell, and
b. expressing the nucleic acid under suitable conditions.

48. An in-vitro process for preparing a human or animal organospecific tissue and/or a human or animal mammalian organ, comprising the following steps:
a. Introducing, into at least one stem cell, one precursor cell and/or one immortalized cell of a human or animal organospecific tissue and/or of a human or animal mammalian organ, in the first place at least one nucleic acid encoding a fusion protein, with the fusion protein containing a wild-type IL-15 and an Fc fragment, and/or at least one vector containing at least one said nucleic acid as claimed in claim 42, and, in the second place, at least one suitable differentiation marker gene,
b. differentiating the cell from step a.,
c. selecting the differentiated cell from step b., and
d. introducing the selected cell from step c. into a human or animal organospecific tissue and/or into a human or animal mammalian organ.
